(19) United States
(12) Patent Application Publication
(10) Pub. No.: US 2025/0064396 A1
(43) Pub. Date: Feb. 27, 2025

(54) WEARABLE RING DEVICE

(71) Applicant: Oura Health Oy, Oulu (FI)

(72) Inventors: Jussi Petteri Järvelä, Kempele (FI); Antti Kalevi Järvelä, Oulu (FI); Ville Tiensuu, Oulu (FI); Tero Juhani Vallius, Kontio (FI)

(21) Appl. No.: 18/797,352

(22) Filed: Aug. 7, 2024

Related U.S. Application Data
(60) Provisional application No. 63/578,094, filed on Aug. 22, 2023.

(51) Int. Cl. A61B 5/00; A61B 5/0205

(52) U.S. Cl. CPC A61B 5/6802; A61B 5/02055; A61B 5/6826

(57) ABSTRACT

Methods, systems, and devices for a wearable ring device are described. A wearable ring device may include a ring-shaped housing and a printed circuit (PCB) that at least partially contacts an inner shell of the ring-shaped housing. Light-emitting components and light-receiving components may be disposed on a first surface of the PCB and may extend through an aperture(s) in the inner shell such that the light-emitting and light-receiving components are substantially flush with an inner ring-shaped surface of the inner shell. Optical lenses may cover the light-emitting and light-receiving components within the aperture(s). In some cases, the optical lenses may be molded over the light-emitting and light-receiving components before the PCB is inserted into the ring-shaped housing. In other cases, the optical lenses may be molded over the light-emitting and light-receiving components (and the aperture(s)) after the PCB is inserted into the ring-shaped housing.

### CROSS REFERENCE

The present Application for Patent claims the benefit of U.S. Provisional Patent Application No. 63/578,094 by JÄRVELÄ et al., entitled “WEARABLE RING DEVICE,” filed Aug. 22, 2023, assigned to the assignee hereof, and expressly incorporated by reference herein.

### FIELD OF TECHNOLOGY

The following relates to wearable devices and data processing, including a form factor for a wearable ring device.

### BACKGROUND

Some wearable devices may be configured to collect data from users including temperature data, heart rate data, and the like. In some cases, the overall structure of the wearable device may affect the accuracy of data measurements performed by the wearable device. Additionally, wearable devices may be intended to be worn full-time, and may therefore be subject to constant wear and tear. As such, there is a desire to improve the durability of wearable devices, while also enabling the wearable devices to be manufactured in an efficient and cost-effective manner.

### BRIEF DESCRIPTION OF THE DRAWINGS

FIG. 1 illustrates an example of a system that supports a form factor for a wearable ring device in accordance with aspects of the present disclosure.

FIG. 2 illustrates an example of a system that supports a form factor for a wearable ring device in accordance with aspects of the present disclosure.

FIG. 3 shows an example of a form factor for a wearable ring device in accordance with aspects of the present disclosure.

FIG. 4 shows an example of a lens molding process that supports a form factor for a wearable ring device in accordance with aspects of the present disclosure.

FIG. 5 shows an example of lens molding processes that supports a form factor for a wearable ring device in accordance with aspects of the present disclosure.

FIGS. 6 through 8 show flowcharts illustrating methods for manufacturing a wearable ring device in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, and the like. As such, some wearable devices may be configured to house one or more sensors configured to acquire physiological data from a user.

In some cases, a wearable device may collect physiological data associated with a user based on skin contact at an optical interface between sensors of the wearable device and the user's skin. In such cases, the structure of a wearable device may affect the ability of the wearable device to efficiently and accurately acquire physiological data. Additionally, wearable devices may be intended to be worn full-time, and may therefore be subject to constant wear and tear. However, space constraints make it difficult to manufacture wearable ring devices with small enough form factors that are comfortable for daily wear. As such, there is a desire to manufacture wearable devices to be durable, while also maintaining the aesthetic appeal of the wearable devices in a small form factor.

Accordingly, aspects of the present disclosure are directed to methods for manufacturing wearable ring devices. In particular, aspects of the present disclosure are directed to processes for manufacturing the structure of the wearable device to reduce the width, thickness, and overall size of the wearable ring device. In particular, aspects of the present disclosure may enable sensors of the wearable device to be moved closer to the user's tissue. By moving the sensors closer to the user's tissue, the thickness of the wearable device may be reduced, and a quality of physiological measurements performed by the sensors may be improved.

In some cases, a wearable ring device may include a ring-shaped housing that includes an inner shell and an outer shell. A printed circuit (PCB) that houses sensors of the device may be encased within the ring-shaped housing between the inner and outer shells. In some cases, light-emitting components and light-receiving components may be disposed on a first surface of the PCB and may extend through one or more apertures in the inner shell such that the light-emitting and light-receiving components are substantially flush with an inner ring-shaped surface of the inner shell. By moving the light-emitting and light-receiving components up into the apertures of the inner shell, the PCB may be moved closer to the inner shell, thereby reducing the thickness of the wearable ring device. Moreover, other electrical components that are not disposed within the aperture(s) (e.g., accelerometers) may be positioned on a second surface of the PCB opposite the first surface, further enabling the PCB to be positioned against the inner shell.

In some aspects, optical lenses may cover the light-emitting and light-receiving components within the one or more apertures of the inner shell. In some cases, the optical lenses may be molded over the light-emitting and light-receiving components before the PCB is inserted into the ring-shaped housing. In other cases, the optical lenses may be molded over the light-emitting and light-receiving components (and the aperture(s)) after the PCB is inserted into the ring-shaped housing. Moreover, the molding process for the optical lenses may be performed separately from a molding process that is used to fill a cavity within the ring-shaped housing and secure the PCB within the cavity.

Aspects of the disclosure are initially described in the context of systems supporting physiological data collection from users via wearable devices. Additional aspects of the disclosure are described in the context of lens molding processes Aspects of the disclosure are further illustrated by and described with reference to apparatus diagrams, system diagrams, and flowcharts that relate to wearable ring devices.

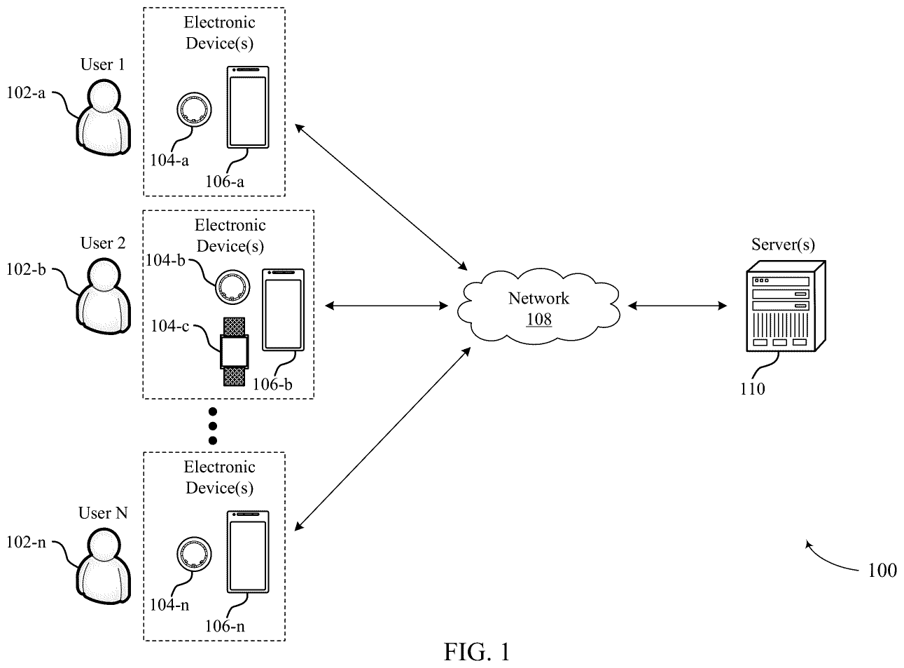

FIG. 1 illustrates an example of a system 100 that supports a form factor for a wearable ring device in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled with one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled with the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Further, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled with one or more servers 110 via wired or wireless communication protocols. For example, as shown in FIG. 1, the electronic devices (e.g., user devices 106) may be communicatively coupled with one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled with the user device 106-a, where the user device 106-a is communicatively coupled with the servers 110 via the network 108. In additional or alternative cases, wearable devices 104 (e.g., rings 104, watches 104) may be directly communicatively coupled with the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

It should be appreciated by a person skilled in the art that one or more aspects of the disclosure may be implemented in a system 100 to additionally or alternatively solve other problems than those described herein. Further, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

In some aspects, the wearable ring devices 104 shown and described in FIG. 1 may be manufactured according to the manufacturing processes described herein in order to reduce the width, thickness, and overall size of the wearable ring devices 104. For example, a wearable ring device 104 of the system 100 may include a ring-shaped housing that includes an inner shell and an outer shell. A PCB that houses sensors of the device may be encased within the ring-shaped housing between the inner and outer shells. In some cases, light-emitting components and light-receiving components may be disposed on a first surface of the PCB and may extend through one or more apertures in the inner shell such that the light-emitting and light-receiving components are substantially flush with an inner ring-shaped surface of the inner shell. By moving the light-emitting and light-receiving components up into the apertures of the inner shell, the PCB may be moved closer to the inner shell, thereby reducing the thickness of the wearable ring device 104.

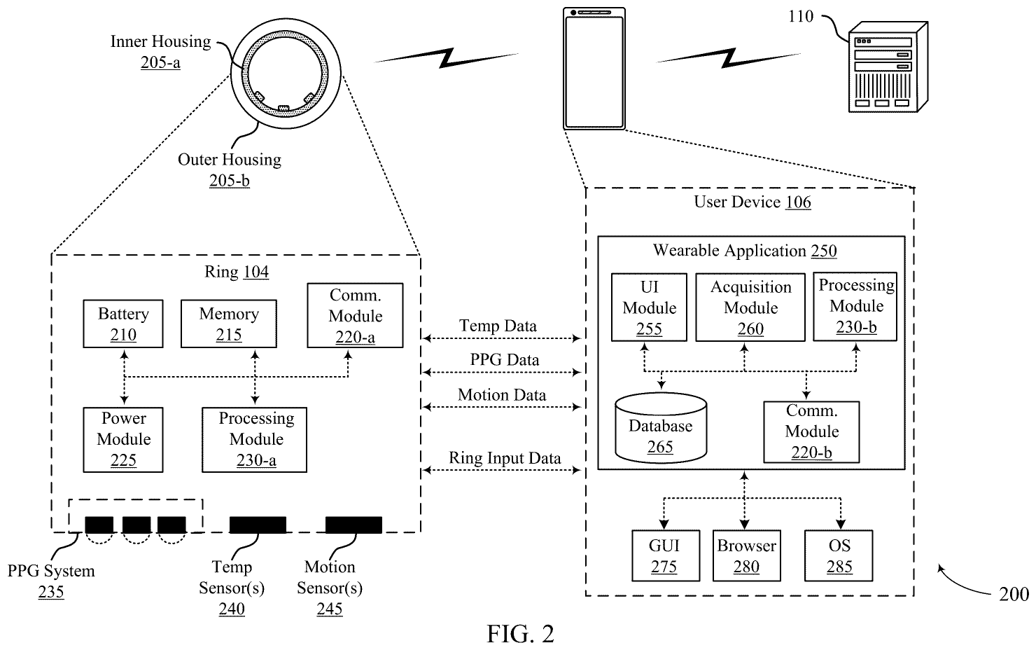

FIG. 2 illustrates an example of a system 200 that supports a form factor for a wearable ring device in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled with one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Further, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled with the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number (e.g., quantity) of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, that may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number (e.g., quantity) and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number (e.g., quantity) of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that rely on relatively low processing power and/or operations that involve a relatively low latency, whereas the user device 106 may transmit the data to the servers 110 for processing operations that rely on relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults may sleep 7-9 hours a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the wearable ring device 104 shown and described in FIG. 2 may be manufactured according to the manufacturing processes described herein in order to reduce the width, thickness, and overall size of the wearable ring devices 104. For example, the wearable ring device 104 of the system 200 may include a ring-shaped housing that includes an inner shell and an outer shell. A PCB that houses sensors of the device may be encased within the ring-shaped housing between the inner and outer shells. In some cases, light-emitting components and light-receiving components may be disposed on a first surface of the PCB and may extend through one or more apertures in the inner shell such that the light-emitting and light-receiving components are substantially flush with an inner ring-shaped surface of the inner shell. By moving the light-emitting and light-receiving components up into the apertures of the inner shell, the PCB may be moved closer to the inner shell, thereby reducing the thickness of the wearable ring device 104.

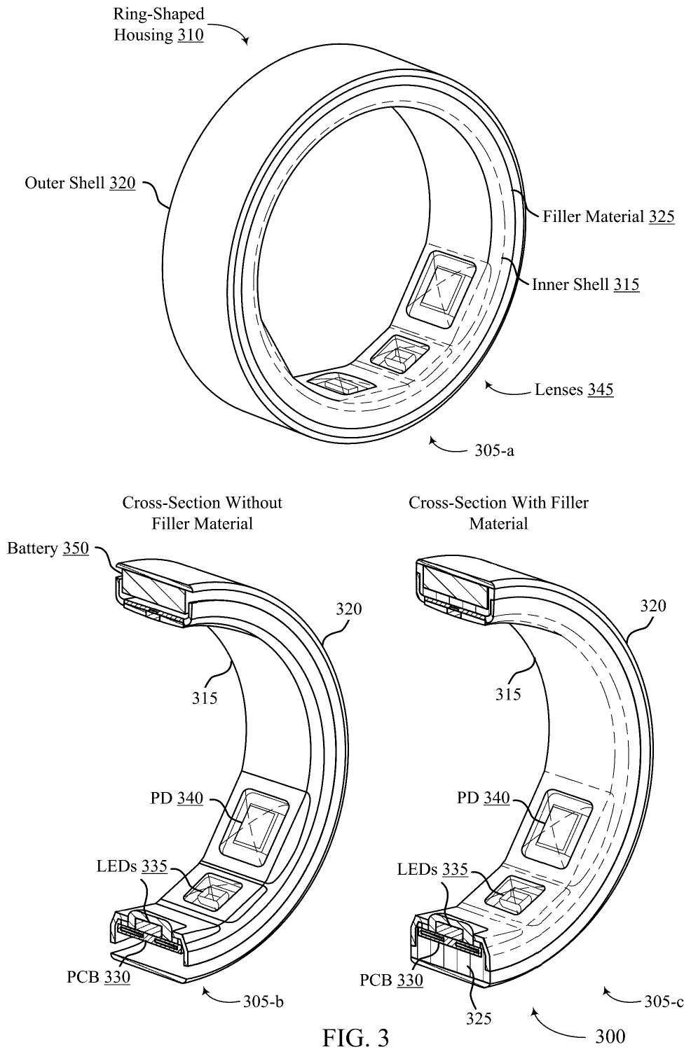

FIG. 3 shows an example of a form factor for a wearable ring device 300 in accordance with aspects of the present disclosure. Aspects of the wearable ring device 300 may implement, or be implemented by, aspects of the system 100, the system 200, or both. For example, the wearable ring device 300 illustrated in FIG. 3 may include an example of the wearable ring devices 104 shown and described with reference to FIGS. 1 and 2.

The wearable ring device 300 is illustrated via three separate views 305 in FIG. 3. The first view 305-a depicts a perspective view of the wearable ring device 300. The second view 305-b depicts a cross-sectional view of the wearable ring device 300 without a filler material (e.g., before a molding process that injects the filler material into the ring). Lastly, the third view 305-c depicts a cross-sectional view of the wearable ring device 300 with the filler material (e.g., after a molding process that injects the filler material into the ring).

As shown in FIG. 3, the wearable ring device 300 may include a ring-shaped housing 310 that includes an inner shell 315 and an outer shell 320, which may be examples of the inner housing 205-a and the outer housing 205-b, respectively. The inner shell 315 may define an inner ring-shaped surface (e.g., inner circumferential surface) of the wearable ring device 300, and the outer shell 320 may define an outer ring-shaped surface of the wearable ring device 300. The inner shell 315 and the outer shell 320 may be made of the same or different materials, such as a metal material (e.g., titanium), a plastic material, an epoxy material, or any combination thereof.

In some cases, a PCB 330 may be positioned within a cavity of the ring-shaped housing 310 between the inner shell 315 and the outer shell 320. As shown in views 305-a and 305-c, a filler material may be injected into the ring-shaped housing to at least partially fill the cavity and secure the PCB 330 to the inner shell 315, the outer shell 320, or both. In such cases, the filler material 325 may secure the outer shell 320 to the inner shell 315. Additionally, or alternatively, the ring-shaped housing 310 may include side covers (e.g., ring-shaped fittings) that are inserted into slots between the inner shell 315 and the outer shell 320, where the side covers (e.g., ring-shaped fittings) bind the inner shell 315 and the outer shell 320 together.

The PCB 330 may include multiple electrical components. For example, the PCB 330 may include one or more light-transmitting components (e.g., LEDs 335) and light-receiving components (e.g., photodetectors (PDs) 340) disposed on a first surface of the PCB 330. The PCB 330 may further include one or more additional electrical components (e.g., accelerometers) disposed on a second surface of the PCB 330 opposite the first surface.

In some aspects, as shown in the first view 305-a, the inner surface of the inner shell 315 may include one or more apertures that are configured to receive the LEDs 335 and PDs 340 of the PCB 330. In some cases, the LEDs 335 and the PDs 340 may extend into the apertures such that a top surface of the LEDs 335 and/or PDs is substantially flush with the inner ring-shaped surface of the inner shell 315. By moving the LEDs 335 and the PDs 340 up into the apertures (instead of being recessed into the apertures), aspects of the present disclosure may enable the PCB 330 to be moved closer to (e.g., make contact with) the inner shell 315, thereby reducing the thickness and size of the wearable ring device 300. Further, moving all other electrical components (aside from components that are expected to be in contact with the user's tissue, such as the LEDs 335, PDs 340, temperature sensors, etc.) to the back-side of the PCB 330 enables the PCB 330 to be moved closer to the inner shell 315, thereby reducing the thickness of the wearable ring device 300.

As shown in the first view 305-a, the inner ring-shaped surface of the inner shell 315 may include a curved region (e.g., region proximate to the curved battery 350), and one or more planar regions. In some cases, the one or more apertures (and therefore the LEDs 335 and PDs 340) may be positioned within or adjacent to the one or more planar regions. Similarly, the PCB 330 may include a curved portion that corresponds to (e.g., is positioned within) the curved region of the inner surface, and one or more planar portions that correspond to (e.g., are positioned within) the curved regions of the inner surface. In such cases, the LEDs 335 and the PDs 340 may be positioned on the planar portions of the PCB 330 such that the LEDs 335 and the PDs 340 align with the apertures disposed within the planar regions of the inner ring-shaped surface of the inner shell 315. In some cases, the planar regions of the inner shell 315 may enable the PCB 330 to be positioned against, or close to, the inner shell 315. That is, the inclusion of planar regions in the inner shell 315 may enable the PCB 330 to be positioned closely against the inner shell 315, particularly around the LEDs 335 and PDs 340. As such, the inclusion of planar regions may help to move the LEDs 335 and PDs 340 further into the apertures, thereby moving the PCB 330 closer to the inner shell 315 and reducing the thickness of the wearable ring device 300. In other cases, the LEDs 335 and PDs 340 may additionally or alternatively be positioned on/within curved regions of the PCB 330/inner shell 315.

In some cases, the PCB 330 may include an adhesive that helps couple the PCB 330 to the inner shell 315. As such, at least a portion of the PCB 330 may be in contact with the inner shell 315. In some cases, the PCB 330 may include a light blocking layer between the LEDs 335 and the PDs 340 that helps prevent stray light from passing from the LEDs 335 and the PDs 340 within the cavity of the ring-shaped housing 310.

In some aspects, the wearable ring device 300 may include one or more optical lenses 345 that cover the LEDs 335 and/or PDs 340. For example, as shown in the first view 305-a, the optical lenses 345 may be positioned within (e.g., substantially fill) the one or more apertures within the inner shell 315. In some cases, the optical lenses 345 may include convex lenses that extend from the inner ring-shaped surface of the inner shell 315. The optical lenses 345 may be formed using a surface mount technology (SMT) process or other molding process.

In some cases, the optical lenses 345 may be formed over the LEDs 335 and the PDs 340 using a separate molding process compared to the molding process used to inject/mold the filler material 325. In this regard, the optical lenses 345 may be made of a different material as compared to the filler material 325. For example, the optical lenses may be made of a (substantially) transparent material that enables light to pass through the one or more apertures, whereas the filler material 325 may include an opaque material. As shown in FIG. 3, the optical lenses 345 may be formed to include one or more facets. In some cases, each of the optical lenses 345 may be formed or molded separately from one another. As such, the shape of each of the optical lenses 345 may be the same or different from one another. For example, the shape of each optical lens 345 may be customized based on whether the optical lens 345 is formed over an LED 335 or PD 340, based on the location of the optical lens 345 within the inner shell 315, based on the size or shape of the respective aperture, or any combination thereof. In this regard, the optical lenses 345 may be tailored to achieve certain optical and/or measurement properties of the wearable ring device 300. For example, the optical lenses 345 may be manufactured separately for each LED 335 and/or PD 340 to help focus light transmitted by the LEDs 335 in certain directions (e.g., toward particular PDs 340, toward certain physiological structures), and to facilitate light reception at the PDs 340 from certain directions (e.g., to minimize stray light).

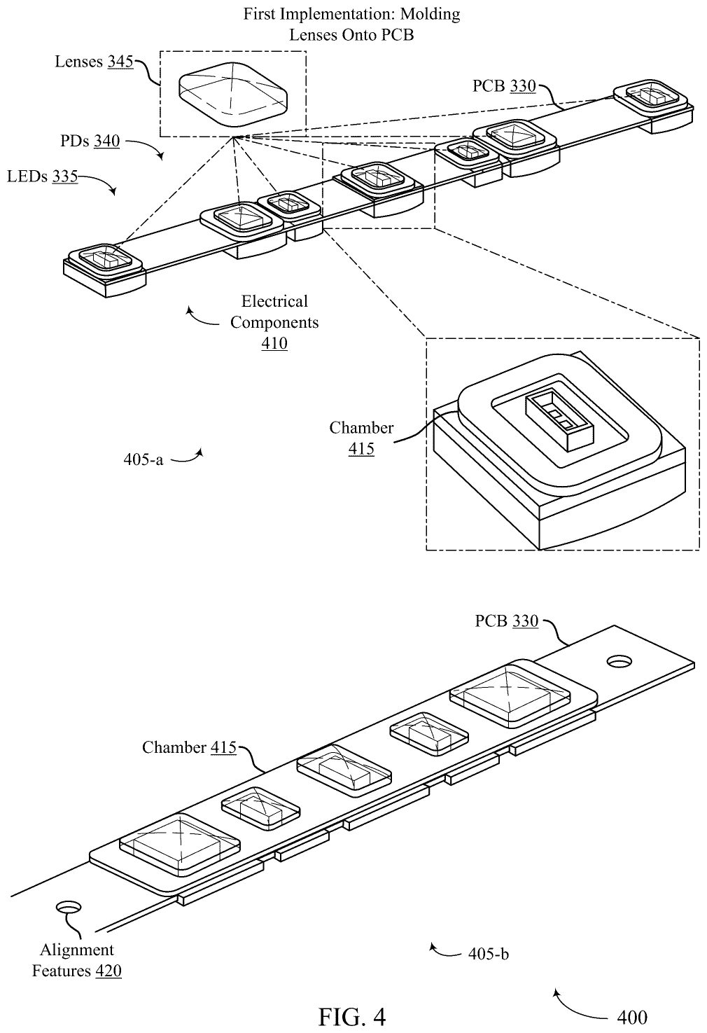

In some aspects, the optical lenses 345 may be formed over the LEDs 335 and the PDs 340 before or after the PCB 330 is inserted into the inner shell 315. For example, in accordance with a first implementation, the optical lenses 345 may be molded onto the PCB 330, where the PCB 330 is then inserted into the inner shell 315 such that the molded/formed optical lenses 345 are inserted into (and substantially fill) the one or more apertures within the inner shell 315. The first implementation for forming the optical lenses 345 will be further shown and described with reference to FIGS. 4-7.

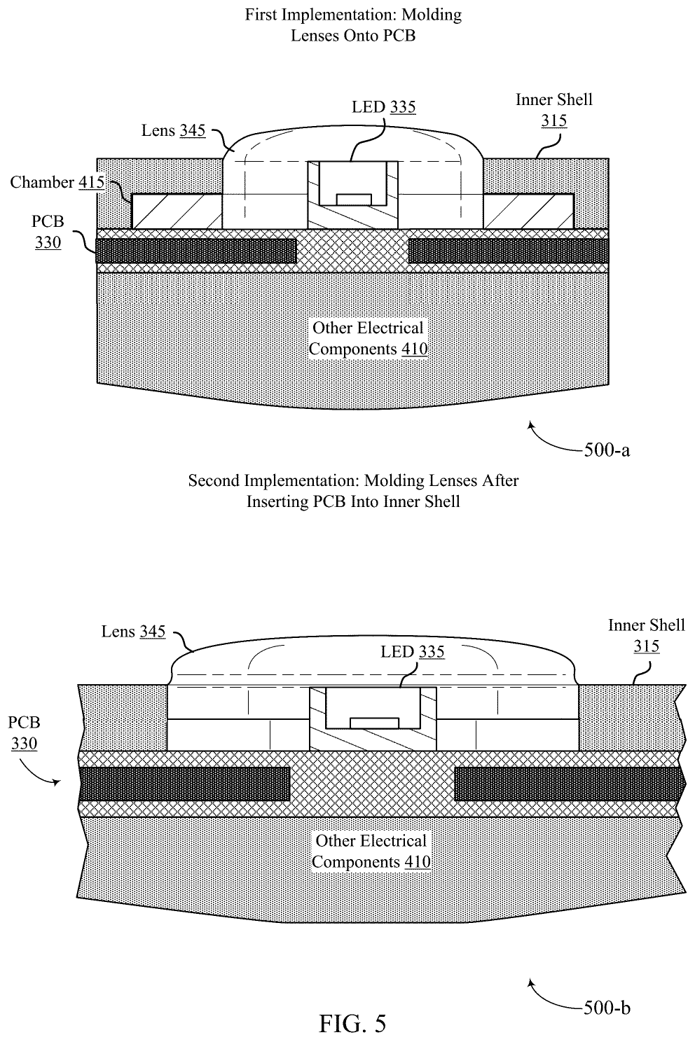

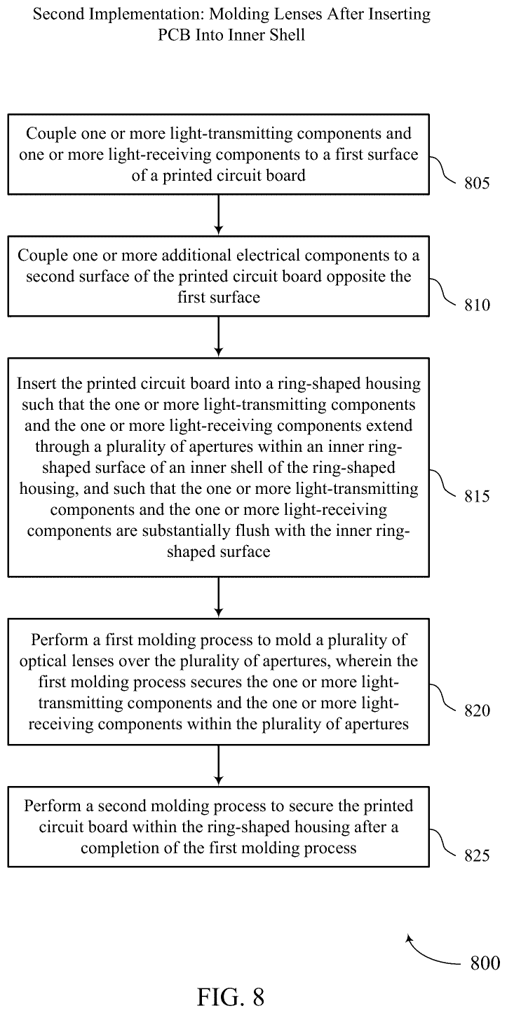

Comparatively, in accordance with the second implementation, the PCB 330 may be coupled with the inner shell 315 such that the LEDs 335 and PDs 340 are inserted into the one or more apertures of the inner shell 315. Subsequently, a molding process may be used to form/mold the optical lenses 345 into/over the apertures and LEDs 335/PDs 340. In this example, the molding process may effectively fill the apertures (and cover the LEDs 335 and PDs 340) with the material used to form the optical lenses 345. The second implementation for forming the optical lenses 345 will be further shown and described with reference to FIGS. 5 and 8.

FIG. 4 shows an example of a lens molding process 400 that supports a form factor for a wearable ring device in accordance with aspects of the present disclosure. Aspects of the lens molding process 400 may implement, or be implemented by, aspects of the system 100, the system 200, the wearable ring device 300, or any combination thereof. In particular, the lens molding process 400 illustrated in FIG. 4 illustrates an example of the first implementation for forming the optical lenses 345 onto the PCB 330.

As shown in FIG. 4, and as described previously herein, the PCB 330 of the wearable ring device 300 may include one or more LEDs 335 and one or more PDs 340 coupled with a first surface of the PCB 330. The PCB 330 may further include one or more additional electrical components 410 coupled with a second surface of the PCB 330 opposite the first surface. As described herein, coupling the LEDs 335 and the PDs 340 (and/or other components that are expected to be in close proximity to the user's tissue, such as temperature sensors) may enable PCB 330 to be in close proximity to the inner shell 315 of the wearable ring device 300 by allowing the LEDs 335 and PDs 340 to be disposed up within the apertures of the inner shell 315. That is, the LEDs 335 and the PDs 340 may be disposed within the apertures, thereby allowing the first surface of the PCB 330 to be in contact with the inner shell 315. Comparatively, other electrical components 410 that are not expected to be in close proximity to the user's tissue, such as accelerometers, gyroscopes, etc., may be positioned on the second surface of the PCB 330.

In some cases, one or more chambers 415 (e.g., frames) may be formed around the LEDs 335 and the PDs 340 on the first surface of the PCB 330. For example, the first view 405-a illustrated in FIG. 4 shows separate chambers 415 formed around each respective LED 335 and PD 340, whereas the second view 405-b shows a single chamber 415 that is collectively formed around the LEDs 335 and PDs 340. The chambers 415 (e.g., frames) may effectively form “wells” where the optical lenses 345 may be formed. In some cases, the chambers 415 (frames) may be formed from a flexible material, such as a soft foam material or shock-absorbing material, to alleviate tolerances during the molding process for the optical lenses 345.

In other cases, the optical lenses 345 may be formed directly onto the surface of the PCB 330 without the use of the chamber(s) 415. In other words, in some cases, the optical lenses 345 may be formed onto the PCB 330 without any physical chamber 415 or frame. In such cases, the filling area for the optical lenses 345 (e.g., the shape/area of the optical lenses 345) may be controlled by other means, such as using materials on the surface of the PCB 330 that exhibit different surface energies, texturing (e.g., lasering), printing, and the like.

As shown in FIG. 4, the optical lenses 345 may be formed over the LEDs 335 and PDs 340. For example, one or more molds may be placed over the chamber(s) 415, where the material for the optical lenses 345 is injected into the molds to substantially fill the chamber(s) 415 and form the optical lenses 345 over the LEDs 335/PDs 340. In some cases, each of the optical lenses 345 may be formed or molded separately from one another. The optical lenses 345 may be formed together or separately (e.g., via one or more separate molding processes). In other words, the mold, molding process, and/or material used to form each optical lens 345 may be the same or different for each optical lens 345. For example, optical lenses 345 may be formed to have different properties, different quantities of facets, and the like. As noted previously herein, forming the optical lenses 345 separately and/or using different molds or materials may enable the optical properties of the LEDs 335, PDs 340, and/or wearable ring device 300 to be customized and tailored, which may lead to improved physiological data collection.

In some cases, the optical lenses 345 may undergo pre-curing using light. In cases where each optical lens 345 is molded separately, each optical lens 345 may be pre-cured using light before the next optical lens 345 is molded. In some aspects, after all optical lenses 345 have been formed (and pre-cured), the wearable ring device 300 may be placed into an oven for final curing with heat.

In accordance with the first implementation for forming the optical lenses 345, after forming the optical lenses 345 onto the PCB 330 (as shown in FIG. 4), the PCB 330 may be coupled with the inner shell 315 of the wearable ring device 300 such that the LEDs 335 and PDs 340 (and therefore the optical lenses 345) are inserted into the one or more apertures of the inner shell 315. The PCB 330 may include one or more alignment features 420 that engage with alignment features within the inner shell 315 in order to ensure that the PCB 330 is oriented correctly within the inner shell 315. In particular, the PCB 330 may be coupled with the inner shell 315 such that the top surface(s) of the LEDs 335 and/or PDs 340 is substantially flush with (or even extend past) the inner ring-shaped surface of the inner shell 315. As such, the optical lenses 345 may extend past the inner-surface of the inner shell 315. Moreover, the optical lenses 345 may be formed to substantially fill an entirety of the apertures. In some cases, the surface of the PCB 330 and/or a surface of the chambers 415 may include an adhesive substance that binds to the inner shell 315 in order to form a water-tight seal around the apertures. In some cases, the inner shell 315 may include one or more recesses surrounding the apertures that are configured to receive the chambers 415 to enable the LEDs 335/PDs 340 to be inserted into the apertures, and to enable the PCB 330 to contact the inner shell 315 around the apertures/lenses 345 (as further shown in FIG. 5). In additional or alternative implementations, the chambers 415 may be removed after forming the optical lenses 345, and prior to attaching the PCB 330 to the inner shell 315.

In accordance with the first implementation, after inserting the PCB 330 with the optical lenses 345 into the inner shell 315, a second molding or injection process may be performed to fill a cavity of the ring-shaped housing 310 with the filler material 325. The filler material 325 may be configured to at least partially fill the cavity and secure the PCB 330 to the inner shell 315, the outer shell 320, or both. Additionally, or alternatively, the PCB 330 may be secured to the inner shell 315, the outer shell 320, or both, through the use of mechanical locking features, side covers, etc., and may therefore be secured within the ring without the use of a second molding process. For example, the PCB 330 may be slid into a groove or other mechanical feature within the inner shell 315 to secure the PCB 330 within the inner shell 315.

FIG. 5 shows an example of lens molding processes 500 that supports a form factor for a wearable ring device in accordance with aspects of the present disclosure. Aspects of the lens molding processes 500-a, 500-b may implement, or be implemented by, aspects of the system 100, the system 200, the wearable ring device 300, the lens molding process 400, or any combination thereof.

In particular, the first lens molding process 500-a illustrates an example of the first implementation for forming the optical lenses 345 onto the PCB 330, as shown and described with reference to FIG. 4. As described previously herein, in accordance with the first implementation, the optical lenses 345 may be formed onto the PCB 330 using one or more chambers 415 prior to coupling the PCB 330 to the inner shell 315. In such cases, the optical lenses 345 may substantially fill the one or more apertures within the inner shell 315, and a surface of the chamber(s) 415 and/or PCB 330 may include an adhesive material or other substance that forms a water-tight seal with the inner shell 315 around the apertures. In some cases, as shown in the first lens molding process 500-a, the inner shell 315 may include one or more recesses surrounding the apertures that are configured to receive the chambers 415 to enable the LEDs 335/PDs 340 to be inserted into the apertures, and to enable the PCB 330 to contact the inner shell 315 around the apertures/lenses 345 (as further shown in FIG. 5). In additional or alternative implementations, the chambers 415 may be removed after forming the optical lenses 345, and prior to attaching the PCB 330 to the inner shell 315.

Comparatively, the second lens molding process 500-b illustrates an example of the second implementation for forming the optical lenses 345 onto the PCB 330. In particular, as compared to the first lens molding process 500-a, where the optical lenses 345 are molded onto the PCB 330 prior to inserting the PCB 330 into the inner shell 315, the second lens molding process 500-b may be performed to form the optical lenses 345 after the PCB 330 has been inserted into the inner shell 315.

For example, referring to the second lens molding process 500-b illustrated in FIG. 5, the PCB 330 may include one or more LEDs 335 and PDs 340 disposed on a first surface of the PCB 330. The PCB may further include other electrical components 410 disposed on a second surface of the PCB 330. In accordance with the first implementation, the PCB m 330 may be inserted into the inner shell 315 such that the surfaces of the LEDs 335/PDs 340 are substantially flush with, or extend past, an inner surface of the inner shell 315. Subsequently, the optical lenses 314 may be formed over the aperture(s) and LEDs 335/PDs 340. For example, a mold may be placed over the aperture of the inner shell 315, and a material may be injected into the mold such that the material substantially fills the aperture and covers the LED 335, thereby forming the optical lens 345. In such cases, the formation of the optical lenses 345 effectively creates a water-tight seal around the aperture(s).

In some implementations, the first molding process for forming the optical lenses 345 may be performed prior to inserting the PCB 330 into the wearable ring device. That is, a mold may be placed over the inner shell 315, and a first molding process may be performed to form the optical lenses 345, where the lenses are formed to include a cavity/recess that is configured to receive the LEDs 335, PDs 340, or other electrical components that are to be positioned within the apertures of the inner shell 315. In such cases, after the optical lenses 345 are formed, the PCB 330 may be coupled with the inner shell 315 such that the LEDs 335 and PDs 340 are inserted into the apertures of the inner shell 315 and into the cavity/recesses formed within the optical lenses 345.

As noted previously herein, the optical lenses 345 may undergo pre-curing using light. In cases where each optical lens 345 is molded separately, each optical lens 345 may be pre-cured using light before the next optical lens 345 is molded. In some aspects, after all optical lenses 345 have been formed (and pre-cured), the wearable ring device 300 may be placed into an oven for final curing with heat.

In accordance with the second implementation, after forming the optical lenses 345 over the apertures of the inner shell 315, a second molding or injection process may be performed to fill a cavity of the ring-shaped housing 310 with the filler material 325. The filler material 325 may be configured to at least partially fill the cavity and secure the PCB 330 to the inner shell 315, the outer shell 320, or both.

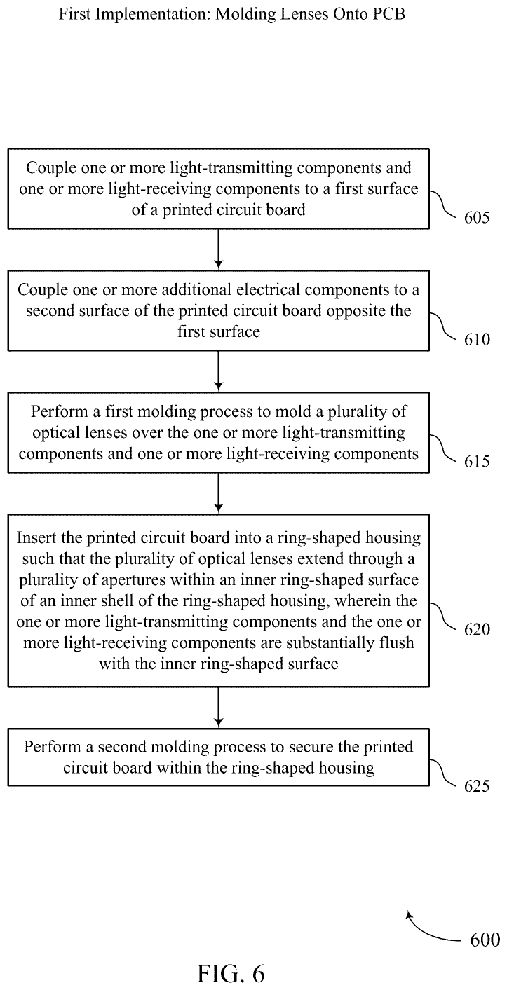

FIG. 6 shows a flowchart illustrating a method 600 for manufacturing a wearable ring device in accordance with aspects of the present disclosure. In particular, the method 600 illustrates an example of the first implementation for forming the optical lenses 345 onto the PCB 330, as shown and described herein. The operations of the method 600 may be implemented by a wearable device or its components as described herein. For example, the operations of the method 600 may be performed by a wearable device as described with reference to FIGS. 1 through 5. In some examples, a wearable device may execute a set of instructions to control the functional elements of the wearable device to perform the described functions. Additionally, or alternatively, the wearable device may perform aspects of the described functions using special-purpose hardware.

At 605, the method may include coupling one or more light-transmitting components and one or more light-receiving components to a first surface of a PCB. The operations of block 605 may be performed in accordance with examples as disclosed herein.

At 610, the method may include coupling one or more additional electrical components to a second surface of the PCB opposite the first surface. The operations of block 610 may be performed in accordance with examples as disclosed herein.

At 615, the method may include performing a first molding process to mold a plurality of optical lenses over the one or more light-transmitting components and one or more light-receiving components. The operations of block 615 may be performed in accordance with examples as disclosed herein.

At 620, the method may include inserting the PCB into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface. The operations of block 620 may be performed in accordance with examples as disclosed herein.

At 625, the method may include performing a second molding process to secure the PCB within the ring-shaped housing. The operations of block 625 may be performed in accordance with examples as disclosed herein.

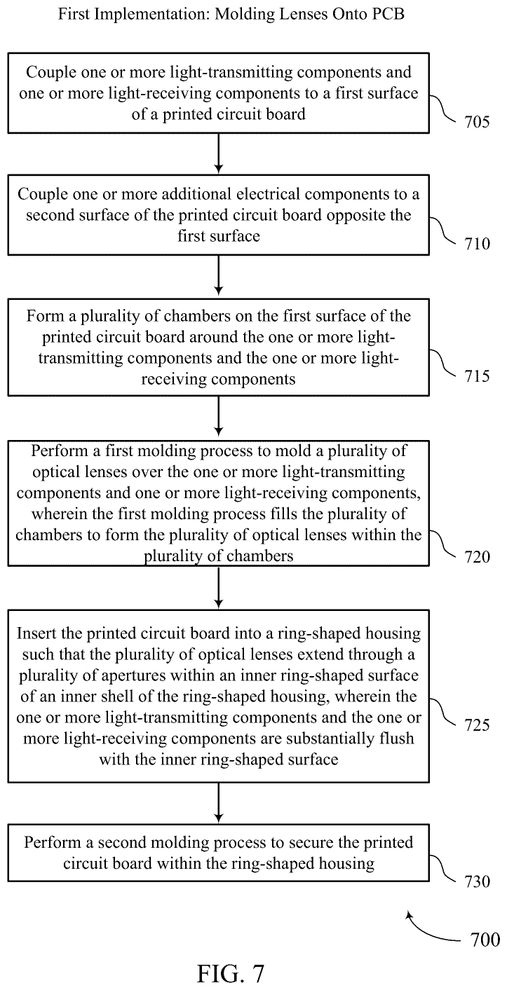

FIG. 7 shows a flowchart illustrating a method 700 for manufacturing a wearable ring device in accordance with aspects of the present disclosure. In particular, the method 700 illustrates an example of the first implementation for forming the optical lenses 345 onto the PCB 330, as shown and described herein. The operations of the method 700 may be implemented by a wearable device or its components as described herein. For example, the operations of the method 700 may be performed by a wearable device as described with reference to FIGS. 1 through 6.

At 705, the method may include coupling one or more light-transmitting components and one or more light-receiving components to a first surface of a PCB. The operations of block 705 may be performed in accordance with examples as disclosed herein.

At 710, the method may include coupling one or more additional electrical components to a second surface of the PCB opposite the first surface. The operations of block 710 may be performed in accordance with examples as disclosed herein.

At 715, the method may include forming a plurality of chambers on the first surface of the PCB around the one or more light-transmitting components and the one or more light-receiving components. The operations of block 715 may be performed in accordance with examples as disclosed herein.

At 720, the method may include performing a first molding process to mold a plurality of optical lenses over the one or more light-transmitting components and one or more light-receiving components, wherein the first molding process fills the plurality of chambers to form the plurality of optical lenses within the plurality of chambers. The operations of block 720 may be performed in accordance with examples as disclosed herein.

At 725, the method may include inserting the PCB into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface. The operations of block 725 may be performed in accordance with examples as disclosed herein.

At 730, the method may include performing a second molding process to secure the PCB within the ring-shaped housing. The operations of block 730 may be performed in accordance with examples as disclosed herein.

FIG. 8 shows a flowchart illustrating a method 800 for manufacturing a wearable ring device in accordance with aspects of the present disclosure. In particular, the method 800 illustrates an example of the second implementation for forming the optical lenses 345 onto the PCB 330, as shown and described herein. The operations of the method 800 may be implemented by a wearable device or its components as described herein. For example, the operations of the method 800 may be performed by a wearable device as described with reference to FIGS. 1 through 5.

At 805, the method may include coupling one or more light-transmitting components and one or more light-receiving components to a first surface of a PCB. The operations of block 805 may be performed in accordance with examples as disclosed herein.

At 810, the method may include coupling one or more additional electrical components to a second surface of the PCB opposite the first surface. The operations of block 810 may be performed in accordance with examples as disclosed herein.

At 815, the method may include inserting the PCB into a ring-shaped housing such that the one or more light-transmitting components and the one or more light-receiving components extend through a plurality of apertures within an inner ring-shaped surface of an inner shell of the ring-shaped housing, and such that the one or more light-transmitting components and the one or more light-receiving components are substantially flush with the inner ring-shaped surface. The operations of block 815 may be performed in accordance with examples as disclosed herein.

At 820, the method may include performing a first molding process to mold a plurality of optical lenses over the plurality of apertures, wherein the first molding process secures the one or more light-transmitting components and the one or more light-receiving components within the plurality of apertures. The operations of block 820 may be performed in accordance with examples as disclosed herein.

At 825, the method may include performing a second molding process to secure the PCB within the ring-shaped housing after a completion of the first molding process. The operations of block 825 may be performed in accordance with examples as disclosed herein.

It should be noted that the methods described herein describe possible implementations, and that the operations and the steps may be rearranged or otherwise modified and that other implementations are possible. Further, aspects from two or more of the methods may be combined.

A method for manufacturing a wearable ring device by an apparatus is described. The method may include coupling one or more light-transmitting components and one or more light-receiving components to a first surface of a PCB, coupling one or more additional electrical components to a second surface of the PCB opposite the first surface, performing a first molding process to mold a plurality of optical lenses over the one or more light-transmitting components and one or more light-receiving components, inserting the PCB into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface, and performing a second molding process to secure the PCB within the ring-shaped housing.

An apparatus for manufacturing a wearable ring device is described. The apparatus may include one or more memories storing processor executable code, and one or more processors coupled with the one or more memories. The one or more processors may individually or collectively operable to execute the code to cause the apparatus to couple one or more light-transmitting components and one or more light-receiving components to a first surface of a PCB, couple one or more additional electrical components to a second surface of the PCB opposite the first surface, perform a first molding process to mold a plurality of optical lenses over the one or more light-transmitting components and one or more light-receiving components, insert the PCB into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface, and perform a second molding process to secure the PCB within the ring-shaped housing.

Another apparatus for manufacturing a wearable ring device is described. The apparatus may include means for coupling one or more light-transmitting components and one or more light-receiving components to a first surface of a PCB, means for coupling one or more additional electrical components to a second surface of the PCB opposite the first surface, means for performing a first molding process to mold a plurality of optical lenses over the one or more light-transmitting components and one or more light-receiving components, means for inserting the PCB into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface, and means for performing a second molding process to secure the PCB within the ring-shaped housing.

A non-transitory computer-readable medium storing code for manufacturing a wearable ring device is described. The code may include instructions executable by a processor to couple one or more light-transmitting components and one or more light-receiving components to a first surface of a PCB, couple one or more additional electrical components to a second surface of the PCB opposite the first surface, perform a first molding process to mold a plurality of optical lenses over the one or more light-transmitting components and one or more light-receiving components, insert the PCB into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface, and perform a second molding process to secure the PCB within the ring-shaped housing.

Some examples of the method, apparatuses, and non-transitory computer-readable medium described herein may further include operations, features, means, or instructions for forming a plurality of chambers on the first surface of the PCB around the one or more light-transmitting components and the one or more light-receiving components, wherein the first molding process fills the plurality of chambers to form the plurality of optical lenses within the plurality of chambers.

In some examples of the method, apparatuses, and non-transitory computer-readable medium described herein, a surface of the plurality of chambers comprises an adhesive that may be configured to secure the plurality of chambers to the inner shell of the wearable ring device and the adhesive creates a water-tight seal around the plurality of apertures.

In some examples of the method, apparatuses, and non-transitory computer-readable medium described herein, the first molding process may be performed with a substantially transparent material and the second molding process may be performed with a second material that may be different from the substantially transparent material.

In some examples of the method, apparatuses, and non-transitory computer-readable medium described herein, the inner ring-shaped surface of the inner shell comprises a curved region and one or more planar regions and the plurality of apertures may be disposed within the one or more planar regions.

In some examples of the method, apparatuses, and non-transitory computer-readable medium described herein, the PCB may be inserted into the ring-shaped housing such that a curved portion of the PCB may be positioned within the wearable ring device proximate to the curved region of the inner shell, and such that one or more planar portions of the PCB may be positioned within the wearable ring device proximate to the one or more planar regions of the inner shell and the one or more light-transmitting components and the one or more light-receiving components may be disposed on the PCB within the one or more planar portions.

In some examples of the method, apparatuses, and non-transitory computer-readable medium described herein, the plurality of optical lenses comprise convex lenses that extend from the inner ring-shaped surface.

In some examples of the method, apparatuses, and non-transitory computer-readable medium described herein, the first molding process may be performed with a substantially transparent material that forms the plurality of optical lenses and inserting the PCB into the ring-shaped housing causes the plurality of optical lenses to substantially fill the plurality of apertures.

A method for manufacturing a wearable ring device by an apparatus is described. The method may include coupling one or more light-transmitting components and one or more light-receiving components to a first surface of a PCB, coupling one or more additional electrical components to a second surface of the PCB opposite the first surface, inserting the PCB into a ring-shaped housing such that the one or more light-transmitting components and the one or more light-receiving components extend through a plurality of apertures within an inner ring-shaped surface of an inner shell of the ring-shaped housing, and such that the one or more light-transmitting components and the one or more light-receiving components are substantially flush with the inner ring-shaped surface, performing a first molding process to mold a plurality of optical lenses over the plurality of apertures, wherein the first molding process secures the one or more light-transmitting components and the one or more light-receiving components within the plurality of apertures, and performing a second molding process to secure the PCB within the ring-shaped housing after a completion of the first molding process.

An apparatus for manufacturing a wearable ring device is described. The apparatus may include one or more memories storing processor executable code, and one or more processors coupled with the one or more memories. The one or more processors may individually or collectively operable to execute the code to cause the apparatus to couple one or more light-transmitting components and one or more light-receiving components to a first surface of a PCB, couple one or more additional electrical components to a second surface of the PCB opposite the first surface, insert the PCB into a ring-shaped housing such that the one or more light-transmitting components and the one or more light-receiving components extend through a plurality of apertures within an inner ring-shaped surface of an inner shell of the ring-shaped housing, and such that the one or more light-transmitting components and the one or more light-receiving components are substantially flush with the inner ring-shaped surface, perform a first molding process to mold a plurality of optical lenses over the plurality of apertures, wherein the first molding process secures the one or more light-transmitting components and the one or more light-receiving components within the plurality of apertures, and perform a second molding process to secure the PCB within the ring-shaped housing after a completion of the first molding process.

Another apparatus for manufacturing a wearable ring device is described. The apparatus may include means for coupling one or more light-transmitting components and one or more light-receiving components to a first surface of a PCB, means for coupling one or more additional electrical components to a second surface of the PCB opposite the first surface, means for inserting the PCB into a ring-shaped housing such that the one or more light-transmitting components and the one or more light-receiving components extend through a plurality of apertures within an inner ring-shaped surface of an inner shell of the ring-shaped housing, and such that the one or more light-transmitting components and the one or more light-receiving components are substantially flush with the inner ring-shaped surface, means for performing a first molding process to mold a plurality of optical lenses over the plurality of apertures, wherein the first molding process secures the one or more light-transmitting components and the one or more light-receiving components within the plurality of apertures, and means for performing a second molding process to secure the PCB within the ring-shaped housing after a completion of the first molding process.

A non-transitory computer-readable medium storing code for manufacturing a wearable ring device is described. The code may include instructions executable by a processor to couple one or more light-transmitting components and one or more light-receiving components to a first surface of a PCB, couple one or more additional electrical components to a second surface of the PCB opposite the first surface, insert the PCB into a ring-shaped housing such that the one or more light-transmitting components and the one or more light-receiving components extend through a plurality of apertures within an inner ring-shaped surface of an inner shell of the ring-shaped housing, and such that the one or more light-transmitting components and the one or more light-receiving components are substantially flush with the inner ring-shaped surface, perform a first molding process to mold a plurality of optical lenses over the plurality of apertures, wherein the first molding process secures the one or more light-transmitting components and the one or more light-receiving components within the plurality of apertures, and perform a second molding process to secure the PCB within the ring-shaped housing after a completion of the first molding process.

In some examples of the method, apparatuses, and non-transitory computer-readable medium described herein, the first surface of the PCB comprises an adhesive that may be configured to secure the PCB to the inner shell of the ring-shaped housing.

In some examples of the method, apparatuses, and non-transitory computer-readable medium described herein, the first molding process may be performed with a substantially transparent material and the second molding process may be performed with a second material that may be different from the substantially transparent material.

In some examples of the method, apparatuses, and non-transitory computer-readable medium described herein, the inner ring-shaped surface of the inner shell comprises a curved region and one or more planar regions and the plurality of apertures may be disposed within the one or more planar regions.

In some examples of the method, apparatuses, and non-transitory computer-readable medium described herein, the PCB may be inserted into the ring-shaped housing such that a curved portion of the PCB may be positioned within the wearable ring device proximate to the curved region of the inner shell, and such that one or more planar portions of the PCB may be positioned within the wearable ring device proximate to the one or more planar regions of the inner shell and the one or more light-transmitting components and the one or more light-receiving components may be disposed on the PCB within the one or more planar portions.

In some examples of the method, apparatuses, and non-transitory computer-readable medium described herein, the plurality of optical lenses comprise convex lenses that extend from the inner ring-shaped surface.

An apparatus of a wearable ring device is described. The apparatus may include a ring-shaped housing comprising an inner shell and an outer shell, wherein the inner shell comprises a plurality of apertures and defines an inner ring-shaped surface of the wearable ring device, and wherein the outer shell defines an outer ring-shaped surface of the wearable ring device, a PCB disposed within the ring-shaped housing, wherein at least a portion of a first surface of the PCB contacts the inner shell, a plurality of electrical components disposed on the PCB, the plurality of electrical components comprising, one or more light-transmitting components and one or more light-receiving components disposed on the first surface of the PCB and extending through the plurality of apertures of the inner ring-shaped surface, wherein the one or more light-transmitting components and one or more light-receiving components are substantially flush with the inner ring-shaped surface, one or more additional components disposed on a second surface of the PCB opposite the first surface, and a plurality of optical lenses covering the one or more light-transmitting components and the one or more light-receiving components disposed within the plurality of apertures.

In some examples of the apparatus, the inner ring-shaped surface of the inner shell comprises a curved region and one or more planar regions, wherein the plurality of apertures may be disposed within the one or more planar regions.

In some examples of the apparatus, the PCB comprises a curved portion positioned within the wearable ring device proximate to the curved region of the inner shell and one or more planar portions positioned within the wearable ring device proximate to the one or more planar regions of the inner shell, wherein the one or more light-transmitting components and the one or more light-receiving components may be disposed on the PCB within the one or more planar portions.

In some examples of the apparatus, the plurality of optical lenses comprise convex lenses that extend from the inner ring-shaped surface.

In some examples of the apparatus, the plurality of optical lenses comprise a substantially transparent material that fills the plurality of apertures.

In some examples of the apparatus, the portion of the first surface comprises an adhesive material that may be configured to couple the portion of the first surface of the PCB to the inner shell.

In some examples of the apparatus, the PCB further comprises a light-blocking layer disposed on the first surface of the PCB between the one or more light-transmitting components and the one or more light-receiving components.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
a ring-shaped housing comprising an inner shell and an outer shell, wherein the inner shell comprises a plurality of apertures and defines an inner ring-shaped surface of the wearable ring device, and wherein the outer shell defines an outer ring-shaped surface of the wearable ring device;
a printed circuit board disposed within the ring-shaped housing, wherein at least a portion of a first surface of the printed circuit board contacts the inner shell;
a plurality of electrical components disposed on the printed circuit board, the plurality of electrical components comprising:
one or more light-transmitting components and one or more light-receiving components disposed on the first surface of the printed circuit board and extending through the plurality of apertures of the inner ring-shaped surface, wherein the one or more light-transmitting components and the one or more light-receiving components are substantially flush with the inner ring-shaped surface; and
one or more additional components disposed on a second surface of the printed circuit board opposite the first surface; and
a plurality of optical lenses covering the one or more light-transmitting components and the one or more light-receiving components disposed within the plurality of apertures.

2. The wearable ring device of claim 1, wherein the inner ring-shaped surface of the inner shell comprises:
a curved region and one or more planar regions, wherein the plurality of apertures are disposed within the one or more planar regions.

3. The wearable ring device of claim 2, wherein the printed circuit board comprises:
a curved portion positioned within the wearable ring device proximate to the curved region of the inner shell; and
one or more planar portions positioned within the wearable ring device proximate to the one or more planar regions of the inner shell, wherein the one or more light-transmitting components and the one or more light-receiving components are disposed on the printed circuit board within the one or more planar portions.

4. The wearable ring device of claim 1, wherein the plurality of optical lenses comprise convex lenses that extend from the inner ring-shaped surface.

5. The wearable ring device of claim 1, wherein the plurality of optical lenses comprise a substantially transparent material that fills the plurality of apertures.

6. The wearable ring device of claim 1, wherein the portion of the first surface comprises an adhesive material that is configured to couple the portion of the first surface of the printed circuit board to the inner shell.

7. The wearable ring device of claim 1, wherein the printed circuit board further comprises:
a light-blocking layer disposed on the first surface of the printed circuit board between the one or more light-transmitting components and the one or more light-receiving components.

8. A method for manufacturing a wearable ring device, comprising:
coupling one or more light-transmitting components and one or more light-receiving components to a first surface of a printed circuit board;
coupling one or more additional electrical components to a second surface of the printed circuit board opposite the first surface;
performing a first molding process to mold a plurality of optical lenses over the one or more light-transmitting components and the one or more light-receiving components;
inserting the printed circuit board into a ring-shaped housing such that the plurality of optical lenses extend through a plurality of apertures within an inner ring-shaped surface of an inner shell of the ring-shaped housing, wherein the one or more light-transmitting components and the one or more light-receiving components are substantially flush with the inner ring-shaped surface; and
performing a second molding process to secure the printed circuit board within the ring-shaped housing.

9. The method of claim 8, further comprising:
forming a plurality of chambers on the first surface of the printed circuit board around the one or more light-transmitting components and the one or more light-receiving components, wherein the first molding process fills the plurality of chambers to form the plurality of optical lenses within the plurality of chambers.

10. The method of claim 9, wherein a surface of the plurality of chambers comprises an adhesive that is configured to secure the plurality of chambers to the inner shell of the wearable ring device, wherein the adhesive creates a water-tight seal around the plurality of apertures.

11. The method of claim 8, wherein the first molding process is performed with a substantially transparent material, and wherein the second molding process is performed with a second material that is different from the substantially transparent material.

12. The method of claim 8, wherein the inner ring-shaped surface of the inner shell comprises a curved region and one or more planar regions, wherein the plurality of apertures are disposed within the one or more planar regions.

13. The method of claim 12, wherein the printed circuit board is inserted into the ring-shaped housing such that a curved portion of the printed circuit board is positioned within the wearable ring device proximate to the curved region of the inner shell, and such that one or more planar portions of the printed circuit board are positioned within the wearable ring device proximate to the one or more planar regions of the inner shell, wherein the one or more light-transmitting components and the one or more light-receiving components are disposed on the printed circuit board within the one or more planar portions.

14. The method of claim 8, wherein the plurality of optical lenses comprise convex lenses that extend from the inner ring-shaped surface.

15. The method of claim 8, wherein the first molding process is performed with a substantially transparent material that forms the plurality of optical lenses, wherein inserting the printed circuit board into the ring-shaped housing causes the plurality of optical lenses to substantially fill the plurality of apertures.

16. A method for manufacturing a wearable ring device, comprising:
coupling one or more light-transmitting components and one or more light-receiving components to a first surface of a printed circuit board;
coupling one or more additional electrical components to a second surface of the printed circuit board opposite the first surface;
inserting the printed circuit board into a ring-shaped housing such that the one or more light-transmitting components and the one or more light-receiving components extend through a plurality of apertures within an inner ring-shaped surface of an inner shell of the ring-shaped housing, and such that the one or more light-transmitting components and the one or more light-receiving components are substantially flush with the inner ring-shaped surface;
performing a first molding process to mold a plurality of optical lenses over the plurality of apertures, wherein the first molding process secures the one or more light-transmitting components and the one or more light-receiving components within the plurality of apertures; and
performing a second molding process to secure the printed circuit board within the ring-shaped housing after a completion of the first molding process.

17. The method of claim 16, wherein the first surface of the printed circuit board comprises an adhesive that is configured to secure the printed circuit board to the inner shell of the ring-shaped housing.

18. The method of claim 16, wherein the first molding process is performed with a substantially transparent material, and wherein the second molding process is performed with a second material that is different from the substantially transparent material.

19. The method of claim 16, wherein the inner ring-shaped surface of the inner shell comprises a curved region and one or more planar regions, wherein the plurality of apertures are disposed within the one or more planar regions.

20. The method of claim 19, wherein the printed circuit board is inserted into the ring-shaped housing such that a curved portion of the printed circuit board is positioned within the wearable ring device proximate to the curved region of the inner shell, and such that one or more planar portions of the printed circuit board are positioned within the wearable ring device proximate to the one or more planar regions of the inner shell, wherein the one or more light-transmitting components and the one or more light-receiving components are disposed on the printed circuit board within the one or more planar portions.
